(19) United States
(12) Patent Application Publication
(10) Pub. No.: US 2007/0298497 A1
(43) Pub. Date: Dec. 27, 2007

(54) Method of Culturing Mesenchymal Stem Cells

(75) Inventor: Glen Delbert Antwiler, Lakewood, CO (US)

Correspondence Address: GAMBRO, INC;PATENT DEPARTMENT, 10810 W COLLINS AVE, LAKEWOOD, CO, 80215, US

(73) Assignee: GAMBRO BCT, INC., Lakewood, CO (US)

(21) Appl. No.: 11/768,657

(22) Filed: Jun. 26, 2007

Related U.S. Application Data
(60) Provisional application No. 60/805,801, filed on Jun. 26, 2006.

(51) Int. Cl. C12N 5/06

(52) U.S. Cl. 435/401

(57) ABSTRACT

This invention is directed toward methods of expanding mesenchymal stem cells ex vivo.

### PRIORITY CLAIM

This application claims priority from U.S. Provisional patent application No. 60/805,801 filed Jun. 26, 2006.

### BACKGROUND

Human stem cells, which have been expanded in culture from a small amount of donor cells, can be used to repair or replace damaged or defective tissues and have broad clinical applications for treatment of a wide range of diseases. Recent advances in the area of regenerative medicine demonstrates that stem cells have unique properties such as self-renewal capacity, the ability to maintain the unspecialized state, and the ability to differentiate into specialized cells under particular conditions.

As an important component of regenerative medicine, the bioreactor or cell expansion system plays an important role in providing optimized environments for cell growth and expansion. The bioreactor provides nutrients to the cells and removal of metabolites, as well as furnishing a physiochemical environment conducive to cell growth in a closed, sterile system.

Many types of bioreactors are currently available. Two of the most common include flat plate bioreactors and hollow fiber bioreactors. Flat plate bioreactors enable cells to grow on large flat surfaces, while hollow fiber bioreactors enable cells to grow either on the inside or outside of the hollow fibers.

Conventional wisdom in the area of stem cell growth and replication has held that in a bioreactor or other ex vivo cell expansion system, adherent cells such as mesenchymal stem cells (MSCs) replicate faster when they are closely surrounded by other MSCs, and therefore a high number of MSCs should be initially loaded into a cell expansion system. Conventional wisdom also has held that the best MSCs for expansion in a bioreactor are those cells which have been highly purified or separated away from other contaminating cell types before being expanded in a bioreactor. The method of this invention teaches that surprisingly, this is not the case.

### BRIEF DESCRIPTION OF THE FIGURES

FIG. 1 is a schematic view of a bioreactor which may be used in this invention.

FIG. 2 is a graph comparing the growth of mesenchymal stem cells having different levels of purity 7 days after reseeding.

FIG. 3 is a graph comparing the growth of mesenchymal stem cells having different levels of purity 14 days after reseeding.

FIG. 4 is another graph comparing the growth of mesenchymal stem cells having different levels of purity.

### SUMMARY OF THE INVENTION

This invention is directed toward a method of expanding mesenchymal stem cells ex vivo including the steps of seeding a collection of cells containing the mesenchymal stem cells on a substrate so that a low density of mesenchymal stem cells adhere to the substrate; expanding the adhered mesenchymal stem cells on the substrate; removing the expanded mesenchymal stem cells from the substrate; and reseeding the mesenchymal stem cells on the same or different substrate.

### DETAILED DESCRIPTION

This invention relates to methods of seeding and expanding MSCs in a cell culturing system.

As discussed above, a number of bioreactor configurations exist for culturing anchorage-dependant cells such as MSCs, and this invention is not dependant upon any particular configuration.

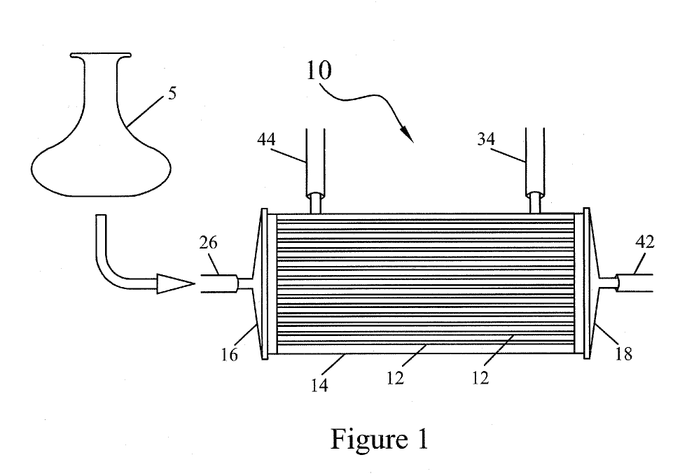

However, as but one example, not meant to be limiting, is a hollow fiber bioreactor shown in FIG. 1. A cell expansion module or bioreactor 10 which may be used in the present invention is made of a bundle of hollow fiber membranes 12 enclosed within a housing 14. The bundle of hollow fibers is collectively referred to as a membrane. The housing or module 14 may be cylindrical in shape and may be made of any type of biocompatible polymeric material.

Each end of the module 14 is closed off with end caps or headers 16, 18. The end caps 16, 18 may be made of any suitable material such as polycarbonate so long as the material is biocompatible with the cells to be grown in the bioreactor.

There may be at least four ports into and out of the module. Two ports fluidly connect to the extracapillary space (EC space), one port 34 for fresh extracapillary media ingress into the space surrounding the hollow fibers and one port 44 for spent extracapillary media egress out of the module. Two ports also fluidly connect to the intracapillary space (IC space), one port 26 for fresh intracapillary media ingress into the lumen of the hollow fibers as well as for the introduction of the cells to be expanded, and one port 42 for spent intracapillary media egress and removal of expanded cells from the bioreactor.

Cells to be expanded in the bioreactor may be flowed into the IC space or the EC space. The bioreactor may be loaded with cells using a syringe or the cells may be distributed into the IC or EC spaces directly from a cell separator. The cells may also be introduced into the growth module or bioreactor from a cell input bag or flask (shown as element 5) which may be sterile docked to the bioreactor.

As discussed above, the number of cells initially seeded in a cell expansion system is thought to be an important factor in how quickly cells expand and reach confluency in a cell expansion system such as the one described above. However, Example 1 below shows that in fact, MSCs do not have to be closely surrounded by other MSCs to achieve optimum growth and expansion.

### EXAMPLE 1

MSCs were initially seeded in flasks at concentrations of 5, 50, 500 and 5000 cells/cm². The time (in hours) it took the cells to double was measured. As shown in Table 1 below, the average doubling time for cells at an initial concentration of 5 cells/cm² is around 35 hours. Cells at a thousand-fold higher concentration took around 123 hours to double. This data suggests that cells initially seeded at lower densities double more quickly than cells initially seeded at higher densities. Lower density is defined as between around 1-100 cells/cm² on a substrate, including between around 5-50 cells/cm² on a substrate.

TABLE 1

| Seeding density MSCs/cm²) | Doubling time (h) |
|---|---|
| 5000 | 123 |
| 500 | 56 |
| 50 | 42 |
| 5 | 35 |

Another factor thought to be important in mesenchymal cell expansion is the purity of the cells initially loaded or seeded in a cell expansion system.

### EXAMPLE 2

To test the theory that to insure maximal expansion of MSCs, MSCs must be purified from other contaminating cells, a four-armed experiment was performed. For the purposes of this and other examples, a purification procedure means any additional procedures performed to remove substantially all contaminating cells from a collection of cells which contain MSCs. The collection of cells could be cells from bone marrow, peripheral blood, umbilical cord, embryonic or any other collection of cells where MSCs reside. Unpurified cells mean any of the above cells not subjected to a purification procedure.

For each arm, after the purification and fractionation step (if performed) the cells were counted. The same number of cells purified (or not) from each arm were plated. The cells were grown until around 1×10⁶ cells were obtained (confluency for the purposes of this example). Confluency is defined as the state at which the cells have expanded so that they cover the entire available surface of the substrate they are being grown on.

For each arm, two sets of experiments were done, one set involved passaging or reseeding the cells every 7 days, and the other set involved passaging or reseeding the cells every 14 days. When the cells reached confluency, the experiments were stopped.

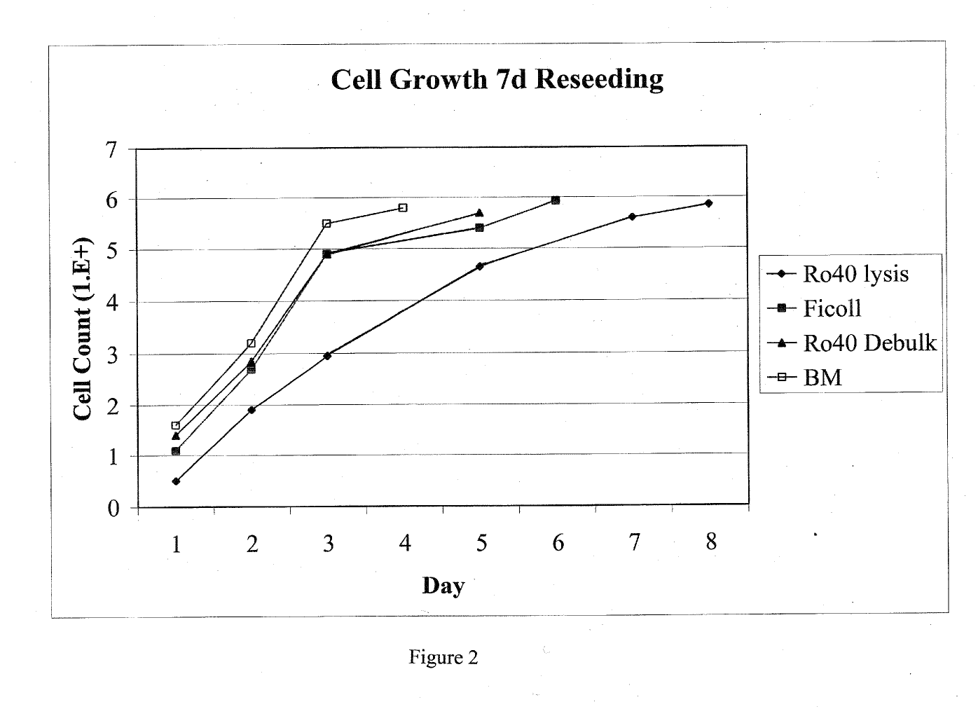

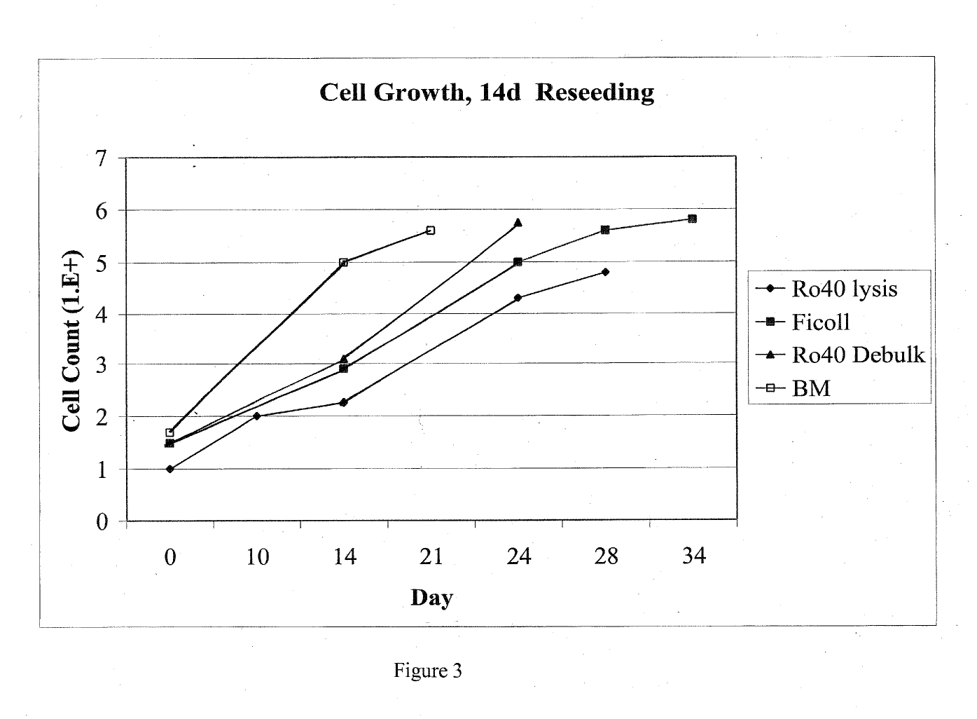

In arm 1, around 50 mL of bone marrow aspirate was removed from a donor and 1 mL was directly plated into a 25 cm² T-flask (designated as BM in FIGS. 2 and 3). No purification or fractionation step was performed. The surface area of a 25 cm² T-flask approximates the surface area of a 1.6 m² hollow fiber bioreactor.

In arm 2, 10 mL of the 50 mL bone marrow aspirate collected from arm 1 was purified to collect the mononuclear cell (MNC) fraction (where stem cells are found) using standard ficoll gradient purification (designated as Ficoll in FIGS. 2 and 3). The fraction was plated onto 25 cm² T-flasks.

Arm 3 involved elutriating MSCs from the remaining 40 mL bone marrow aspirate collected in arm 1. MSCs were debulked of contaminating red blood cells and elutriated using the Elutra system (available from Gambro BCT, Inc., Lakewood, Colo., USA) (designated as Ro40 Debulk in FIGS. 2 and 3).

In arm 4, the remaining unplated fraction from arm 3 was subjected to a lysing procedure to remove any remaining contaminating red blood cells (designated as Ro40 lysis in FIGS. 2 and 3).

Representative graphs are shown in FIGS. 2 and 3. As can be seen, the mesenchymal stem cells which were reseeded every 7 days (FIG. 2) reached confluency at a faster rate then the cells which were reseeded every 14 days (FIG. 3). Surprisingly, the cells which received the least amount of manipulation (directly plated bone marrow containing contaminating cells) were the fastest growing. Cells which received the greatest manipulation, and thus were assumed to have the greatest purity, (elutriated MSCs with any contaminating red blood cells lysed) were the slowest growing.

These findings also suggest that MSCs which were reseeded before reaching confluency grow at least as fast as the MSCs which were seeded initially after collection. This finding might imply that the reseeding process has no adverse effect on MSC growth in a bioreactor, and that reusing (reseeding) the same surface the cells were initially seeded on appears to have no adverse effect on the growth of the cells. However, the cells could be reseeded on a different substrate as well.

It is therefore desirable not to grow MSCs to confluency. Cells should be harvested well before a confluent state is reached. The growth of enough cells to provide a sufficient therapeutic dose of MSCs would be reached quickly.

### EXAMPLE 3

In another example, a standard elutriation protocol such as that described in U.S. patent application Ser. No. 11/131063 was used to enrich subsets of stem cells from bone marrow to determine how cells which were more purified would grow as compared to cells which were not purified away from contaminating cells.

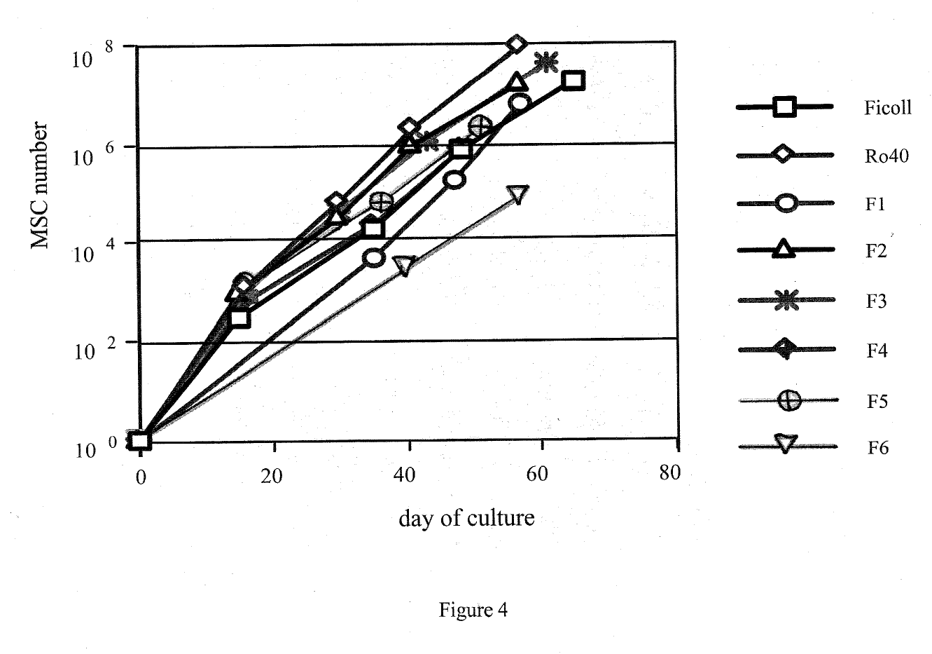

Six elutriated fractions of bone marrow containing substantially purified MSCs were grown to confluency. Substantially purified means that the majority of cells in a particular elutriated fraction are the same cell type. These elutriated fractions are shown as F1-F6 in FIG. 4. A 50 mL sample of bone marrow was also purified using the commonly used purification procedure of a Ficoll gradient for comparison (shown as Ficoll in FIG. 4). 40 rotor off (shown as Ro40 in FIG. 4) means that after the plasma, platelets and red blood cells were removed from the initial 50 mL bone marrow sample, the rotor was turned off and all the cells remaining in the 40 mL elutriation chamber were collected. This procedure does not elute individual fractions or subsets of cells.

FIG. 4 compares the time in days for the cells from the individual elutriated fractions as well as the ficoll purified cells and the cells collected with the rotor off to expand to 1×10⁸ cells. As can be seen, fractions F1-F6 containing the most substantially purified cells grew the slowest, while the least purified cells (rotor off or Ro40) grew the fastest. This corroborates the findings in Example 2 above, that cells which are the least purified, grow the fastest.

### EXAMPLE 4

As discussed above, the purity of the MSCs to be expanded is thought to effect how quickly the cells expand in culture.

Example 4 compares the alpha (a), or time in days it takes for cells to double, of unpurified MSCs in 50 mLs of bone marrow which was directly seeded into a hollow fiber bioreactor, with MSCs which were pre-selected before being seeded into the bioreactor. Direct seeding means that bone marrow taken from a patient was put directly into a bioreactor without removing any contaminating cells such as platelets and red blood cells. Bone chips and other non-cellular matter may be removed. Pre-selected means that 50 mLs of bone marrow was initially plated on a T-75 flask and the MSCs contained therein were allowed to attach to the flask and grow to confluency before being reseeded in a hollow fiber bioreactor. Pre-selection ensures that all of the cells to be expanded in the bioreactor are pure MSCs. Two types of hollow fiber membranes, a 0.5% thermoplastic polyurethane sold under the trade name Desmopan® (available from Bayer MaterialScience AG, DE) and Polyflux® (a blend of polyamide, polyarylethersulfone and polyvinylpyrrolidone) (available from Gambro Dialysatoren, GmBH, Hechingen, Del.) were tested. Both Desmopan and Polyflux are synthetic membranes.

TABLE 2

| | | Bioreactor with pre- |
|---|---|---|
| | Direct seeded bioreactor | selected MSCs |
| | α | α |
| 0.5% Desmopan | 17.3 | 26.7 |
| Polyflux | 16.8 | 23.7 |

As is seen from Table 2 above, MSCs which were directly seeded into a bioreactor with other contaminating cells in the bone marrow doubled at a faster rate (took less days to double) than MSCs which were purified first before being expanded in a bioreactor. This is true for both types of synthetic membranes.

As shown in the above examples, to achieve faster MSC expansion in a cell expansion system, the cells need not be purified away from other contaminating cells. MSCs should also be reseeded often and not be allowed to reach confluency before being reseeded.

1. A method of expanding mesenchymal stem cells ex vivo comprising the steps of:
a.) seeding a collection of cells containing the mesenchymal stem cells on a substrate so that a low density of mesenchymal stem cells adhere to the substrate;
b.) expanding the adhered mesenchymal stem cells on the substrate;
c.) removing the expanded mesenchymal stem cells from the substrate;
d.) reseeding the removed mesenchymal stem cells on the same or different substrate; and
e.) repeating steps b)-d) until the desired number of expanded mesenchymal stem cells is reached.

2. The method of claim 1 wherein the low density of mesenchymal stem cells is between around 1-100 mesenchymal stem cells/cm².

3. The method of claim 1 wherein the low density of mesenchymal stem cells is between around 5-50 mesenchymal stem cells/cm².

4. The method of claim 3 wherein the low density of mesenchymal stem cells further comprises a density of around 5 mesenchymal stem cells/cm².

5. The method of claim 3 wherein the low density of mesenchymal stem cells further comprises a density of around mesenchymal stem 50 cells/cm².

6. The method of claim 1 wherein the step of reseeding the mesenchymal stem cells further includes reseeding the mesenchymal stem cells before they reach confluency.

7. The method of claim 1 wherein the step of seeding further comprises seeding the collection of cells containing the mesenchymal stem cells prior to performing a purification procedure.

8. The method of claim 1 wherein the step of seeding further comprises seeding bone marrow cells containing the mesenchymal stem cells.

9. The method of claim 1 wherein the step of seeding further comprises seeding peripheral blood cells containing the mesenchymal stem cells.

10. The method of claim 1 wherein the step of seeding further comprises seeding umbilical cord cells containing the mesenchymal stem cells.

11. The method of claim 1 wherein the step of seeding further comprises seeding embryonic cells containing the mesenchymal stem cells.

12. The method of claim 1 wherein the step of seeding further comprises seeding on a synthetic membrane.

13. The method of claim 12 wherein the synthetic membrane is Polyflux membrane.

14. The method of claim 12 wherein the synthetic membrane is Desmopan membrane.

15. The method of claim 12 wherein the method of seeding on a synthetic membrane further comprises seeding in hollow fibers.

16. A method of expanding mesenchymal stem cells ex vivo comprising the steps of:
a.) seeding a collection of unpurified cells containing the mesenchymal stem cells on a substrate so that the mesenchymal stem cells adhere to the substrate; and
b.) expanding the adhered mesenchymal stem cells on the substrate.

17. The method of claim 16 wherein the step of seeding further comprises seeding unpurified bone marrow cells containing the mesenchymal stem cells.

18. The method of claim 16 wherein the step of seeding further comprises seeding unpurified peripheral blood cells containing the mesenchymal stem cells.

19. The method of claim 16 wherein the step of seeding further comprises seeding unpurified umbilical cord cells containing the mesenchymal stem cells.

20. The method of claim 16 wherein the step of seeding further comprises seeding unpurified embryonic cells containing the mesenchymal stem cells.

21. The method of claim 16 wherein the step of seeding further comprises seeding on a synthetic membrane.

22. The method of claim 21 wherein the synthetic membrane is Polyflux membrane.

23. The method of claim 21 wherein the synthetic membrane is Desmopan membrane.

24. The method of claim 21 wherein the method of seeding on a synthetic membrane further comprises seeding in hollow fibers.
